(19) United States
(12) Patent Application Publication
(10) Pub. No.: US 2021/0228118 A1
(43) Pub. Date: Jul. 29, 2021

(54) APPARATUS AND METHOD FOR MONITORING BRAIN ACTIVITY

(71) Applicant: AXEM NEUROTECHNOLOGY INC., Halifax (CA)

(72) Inventors: Christopher Friesen, Halifax (CA); Tony Joseph Gerald Ingram, Halifax (CA); Eric Anthony Hamilton, Halifax (CA); Michael Lawrence, Halifax (CA); Karl Ryland Leuschen, Brookside (CA); Megan Melissa Smith, Nine Mile Road (CA); Luke Murphy Haliburton, Antigonish (CA); Chad Douglas Murphy, Porters Lake (CA)

(21) Appl. No.: 17/256,469

(22) PCT Filed: Jul. 5, 2019
(86) PCT No.: PCT/CA2019/050935

§ 371 Date: Dec. 28, 2020

Related U.S. Application Data
(60) Provisional application No. 62/694,447, filed on Jul. 6, 2018.

(51) Int. Cl. A61B 5/1455; A61B 5/00

(52) U.S. Cl. CPC A61B 5/14553; A61B 5/14552; A61B 5/6803; A61B 5/6843; A61B 5/7278; A61B 5/02433; A61B 2562/043; A61B 2562/0242; A61B 2562/228; A61B 2562/223; A61B 2562/0238

(57) ABSTRACT

A method and apparatus for monitoring brain activity of a user is disclosed. The apparatus includes a plurality of spatially separated emitters operable to generate infrared radiation. The apparatus also includes a plurality of spatially separated infrared radiation detectors, and a plurality of light pipes urged into contact with the user's scalp, each one of the plurality of emitters and detectors having an associated light pipe operable to couple infrared radiation from the emitter into the scalp or to couple infrared radiation from the scalp to the detector. Each detector is operable to produce a signal representing an intensity of infrared radiation generated by a selectively actuated one of the plurality of emitters and received at the detector after traveling on a path through underlying brain tissue, the signals being received by a controller operably configured to process the signals from each detector to determine changes in blood oxygenation within the brain tissue along the path between the respective emitter and detector, and generate a spatial representation of brain activity within in the user's brain based on the processed signals.

### RELATED APPLICATIONS

This application claims the benefit of provisional patent application 62/694447 entitled “SYSTEM AND METHOD FOR A USABLE DEVICE FOR MONITORING BRAIN ACTIVITY”, filed on Jul. 6, 2018 and incorporated herein by reference in its entirety.

### BACKGROUND

### 1. Field

This disclosure relates generally to brain activity monitoring and more particularly to brain activity monitoring using infrared light.

### 2. Description of Related Art

Near-infrared Spectroscopy may be used to measure brain activity in the motor cortex by measuring relative changes in oxygen concentration in the brain. Brain activity requires oxygen to use energy, which is known as the hemodynamic response and is the basis for many brain imaging technologies. When a user moves their left hand, the concentration of oxygen will increase in the right motor cortex in the area that controls the hand. The more muscle recruitment and the more complex the movement, the greater the oxygen change.

Individuals with an acquired brain injury (such as a stroke) often have mobility impairments, requiring intensive physical rehabilitation. Rehabilitation promotes recovery by leveraging neuroplasticity (i.e. the brain's ability to change). Brain activity metrics may be used to predict recovery, track progress, and compare the effects of different exercises, potentially allowing clinicians to better tailor therapy to individual patients. There remains a need for brain activity monitoring methods and apparatus.

### SUMMARY

In accordance with one disclosed aspect there is provided an apparatus for monitoring brain activity of a user. The apparatus includes a plurality of spatially separated emitters operable to generate infrared radiation. The apparatus also includes a plurality of spatially separated infrared radiation detectors, and a plurality of light pipes urged into contact with the user's scalp, each one of the plurality of emitters and detectors having an associated light pipe operable to couple infrared radiation from the emitter into the scalp or to couple infrared radiation from the scalp to the detector. Each detector is operable to produce a signal representing an intensity of infrared radiation generated by a selectively actuated one of the plurality of emitters and received at the detector after traveling on a path through underlying brain tissue, the signals being received by a controller operably configured to process the signals from each detector to determine changes in blood oxygenation within the brain tissue along the path between the respective emitter and detector, and generate a spatial representation of brain activity within in the user's brain based on the processed signals.

The emitters and detectors are disposed on a headset and the controller may be remotely disposed with respect to the headset and the headset may include a transmitter operable to transmit the signals to the controller for processing.

The apparatus may include a headset controller disposed on the headset and operably configured to control functions of the transmitter, the emitters, and the detectors.

The infrared radiation may include near infrared radiation.

The emitter may include a light emitting diode operably configured to produce the infrared radiation at a plurality of wavelengths selected to cause the detector to produce signals that facilitate determination of a blood oxygenation state of the brain tissue underlying each of the spatially separated emitters and associated detectors, the blood oxygenation state being indicative of local cerebral hemodynamics within the brain tissue and facilitating a determination of neural activity within the user's brain.

The plurality of wavelengths may include at least first and second wavelengths selected to fall on either side of the isobestic point for oxygenation and deoxygenation of blood hemoglobin.

The light emitting diode associated with each of the plurality of emitters may be mounted within a headset, the headset being operable to support the plurality of emitters and plurality of detectors in contact with the user's scalp when worn by the user.

The plurality of emitters may include at least one emitter disposed proximate to one of the plurality of detectors and the detector may be operable to produce a shallow path signal representing an intensity of infrared radiation generated after traveling along a shallow path through scalp and bone tissue between the at least one emitter and the detector, at least one emitter disposed spaced apart from one or more of the plurality of detectors and the one or more detectors are operable to produce a deep path signal representing an intensity of infrared radiation generated after traveling along a deep path through the underlying brain tissue between the at least one emitter and the one or more detectors.

The controller may be operably configured to process the shallow path signals to determine shallow path noise, the shallow path noise being used as a basis for filtering the deep path signal to determine the changes in blood oxygenation within the brain tissue.

The controller may be operably configured to process the shallow path signals to determine shallow path noise, the shallow path noise being used as a basis for filtering the deep path signal to determine the changes in blood oxygenation within the brain tissue.

The controller may be operably configured to process the signals by aligning a phase of each of the shallow path signals and deep path signals based on a physiological process component in the signals, performing a principle component analysis on the shallow path signals to determine contamination components associated with physiological processes other than changes in blood oxygenation within the brain tissue, and removing the contamination components from the deep path signals to provide signals representing changes in blood oxygenation within the brain tissue from which the effects of other physiological processes have been filtered.

Performing the principle component analysis may include filtering the shallow path signals to separate the shallow path signals into slow-cycling signals associated with slow-cycling physiological processes and fast-cycling signals associated with fast-cycling physiological processes and performing principle component analysis on each of the shallow path signals, the slow-cycling signals and the fast-cycling signals.

The controller may be operably configured to, prior to performing the principle component analysis, process the phase aligned shallow path signals to generate signals representing oxygenation and deoxygenation of blood hemoglobin, and take a first derivative of the signals representing oxygenation and deoxygenation of blood hemoglobin.

The controller may be operably configured to activate selected emitters and detectors to generate signals associated with different paths of travel of the infrared radiation through the brain tissue.

Each light pipe may include a low durometer material that is optically transmissive at wavelengths associated with the infrared radiation, the low durometer material facilitating comfortable optical contact with the scalp of the user.

The light pipe material may have a durometer in a range of between about Shore A durometer 30 and about Shore A durometer 90.

The length of each light pipe may be between about 7 millimeters and 15 millimeters.

Each of the plurality of emitters and detectors may be mounted on a headset that conforms to the scalp of the user and a length of at least about 7 mm of the light pipe may protrude outwardly from a surface of the headset.

Each light pipe may include a coupling surface for coupling infrared radiation between the light pipe and the emitter or detector, a distal lens operably configured to contact the scalp and direct infrared radiation to or from the light pipe, and a guide portion extending between the coupling surface and the distal lens.

The apparatus may include a sheath surrounding at least a portion of the guide portion of each light pipe, the sheath being operably configured to reduce infrared radiation leakage from the guide portion of the light pipe.

The sheath may include an outer surface operably configured to divert the user's hair away from the distal lens when the light pipe is in contact with the scalp.

The guide portion of the light pipe may have a generally cylindrical shape and may have a diameter selected to cause total internal reflection of infrared radiation incident at inner surfaces of the guide portion.

The coupling surface of the light pipe may be operably configured to directly contact a radiating surface of the emitter or a radiation receiving surface of the detector for coupling infrared radiation between the light pipe and the detector.

A cross sectional area of the guide portion may be smaller than a cross sectional area of the coupling surface and the light pipe may further include a tapered transition between the coupling surface and the guide portion and a taper angle of the tapered transition may be selected to prevent infrared radiation leakage from the tapered transition, the tapered transition further providing for mounting of the light pipe to the emitter or detector.

The apparatus may include a headset having a plurality of articulated segments, each articulated segment supporting at least one emitter or detector, the articulated segments each being urged toward the scalp of the user to cause contact between the associated light pipes of the respective emitters or detectors and the scalp.

Each of the plurality of articulated segments may be operably configured to mount a circuit substrate and at least one detector or emitter may be mounted on each circuit substrate.

The apparatus may include a flexible interconnect interconnecting between a headset controller and the plurality of circuit substrates.

The flexible interconnect and the plurality of circuit substrates may be formed as a unitary flexible circuit substrate.

The plurality of detectors are disposed spaced apart along a sprung band having a curvature operable to conform to a corresponding lateral curvature of the user's scalp and urge the plurality of detectors toward the scalp when the band is worn by the user.

The apparatus may further include a plurality of articulated segments disposed forwardly or rearwardly with respect to the sprung band, each articulated segment including at least one emitter and being urged toward the scalp when the band is worn by the user.

The controller may be operably configured to monitor the signal level produced at each detector and to control a level of infrared radiation produced by the selectively actuated emitter to maintain the intensity within a detection range of the detector.

The controller may be further operably configured to generate display data for display as a graphic user interface (GUI) on a screen in communication with the controller, the GUI including a spatial representation of at least one of the emitters and detectors along with display information indicating whether the signal intensity is within the detection range of the associated detector.

The controller may be operably configured to discontinue the monitoring when the signals received from the detectors no longer meet a coupling criterion indicative of a plurality of the emitters or detectors being coupled to the scalp of the user.

The apparatus may include at least one coupling sensor operably configured to generate a coupling signal indicating a state of coupling between the plurality of light pipes and the user's scalp, and the controller may be operably configured to discontinue the monitoring in response to the coupling signal indicating that a coupling criterion is not being met.

The at least one coupling sensor may include at least one of a capacitive sensor that produces a signal indicative of a proximity of the apparatus to the scalp, an acoustic sensor that produces a signal in response to an ambient sound level, an inertial sensor that produces a signal indicative of movement of the apparatus, or one or more of the detectors, an ambient light component in the signal produced by the one or more detectors may be indicative of the apparatus being removed from the scalp and the detector being subject to ambient light radiation.

The controller may be operably configured to process the signal received by at least one of the detectors to extract a cardiac pulse signal representing a detected heartbeat of the user and to monitor the pulse signal to determine whether coupling between the emitters and detectors and the scalp of the user meets a coupling criterion.

The controller may be operably configured to process the signals by extracting a dominant frequency from the signals that falls within a frequency range based on the user's expected heartbeat frequency range.

The controller may be operably configured to discontinue the monitoring when the cardiac pulse signals received from the detectors no longer meet the coupling criterion.

The controller may be operably configured to monitor time variations in blood oxygenation within the brain tissue in a region underlying each detector and selectively actuated emitter and to generate data metrics representing a degree of brain activation in each region.

The controller may be further operably configured to generate display data for display as a graphic user interface (GUI) on a screen in communication with the controller, the GUI including a representation of regions of the user's body that correspond to regions of the user's brain that are indicated by the changes in blood oxygenation within the brain tissue to be actuated.

The controller may include a processor circuit, the processor circuit including a graphic processing unit operably configured to accelerate processing of the signals from each of the plurality of detectors to facilitate near real time presentation of results to the user.

Other aspects and features will become apparent to those ordinarily skilled in the art upon review of the following description of specific disclosed embodiments in conjunction with the accompanying figures.

### BRIEF DESCRIPTION OF THE DRAWINGS

In drawings which illustrate disclosed embodiments,

FIG. 1 is a perspective view of an apparatus for monitoring brain activity in accordance with a first disclosed embodiment;

FIG. 2A is an exploded view of an emitter enclosure of the apparatus shown in FIG. 1;

FIG. 2B is an elevational view of an emitter and light pipe of the apparatus shown in FIG. 1;

FIG. 3 is an exploded view of a detector enclosure of the apparatus shown in FIG. 1

FIG. 4 is a plan view of a headset shown in FIG. 1;

FIG. 5 is an exploded view of portions of the headset shown in FIG. 4;

FIG. 6 is a block diagram of a headset controller and a host controller of the apparatus shown in FIG. 1;

FIG. 7A is a process flowchart depicting blocks of code for directing the headset controller shown in FIG. 6 to perform a signal calibration process;

FIG. 7B is a process flowchart depicting blocks of code for directing the headset controller shown in FIG. 6 to acquire signals;

FIG. 8 is a process flowchart depicting blocks of code for directing a processor circuit of the host controller to perform an assessment session;

FIG. 9A-9D are a series of screenshots showing display screens generated and displayed on a display of the host controller; and

FIG. 10 is a process flowchart depicting blocks of code for directing the host controller to implement data signal processing functions shown in FIG. 8.

### DETAILED DESCRIPTION

### System Overview

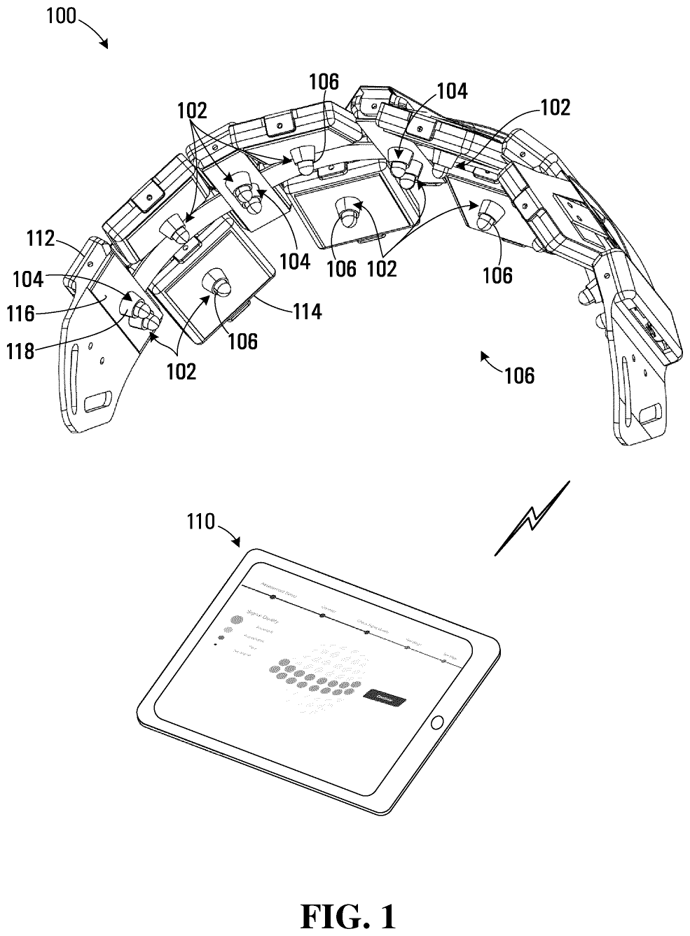

Referring to FIG. 1, an apparatus for monitoring brain activity through a user's scalp according to a first disclosed embodiment is shown generally at 100. The apparatus 100 includes a plurality of spatially separated near infrared radiation emitters 102 and a plurality of spatially separated near infrared radiation detectors 104. Each one of the emitters 102 and the detectors 104 have an associated light pipe 106, which is operable to couple near infrared radiation from the emitter into the user's scalp or to couple near infrared radiation from the scalp to the detector. In this embodiment the emitters 102 are mounted within a headset 108 operable to support the plurality of emitters 102 and plurality of detectors 104 in contact with the user's scalp when the headset is worn by the user such that each of the light pipes 106 contact the user's scalp.

Each detector 104 is operable to produce a signal representing an intensity of near infrared radiation generated by a selectively actuated one of the plurality of emitters 102 and received at the detector after traveling on a path through underlying brain tissue. Near infrared radiation (i.e. near infrared light) has a wavelength generally within a range of about 750 nm (nanometers) to 900 nm and is able to travel through skin, tissue, and bone. The near infrared radiation from each emitter 102 thus penetrates the scalp and skull and travels along a path through respective portions of underlying brain tissue, which reflects the radiation back to one or more of the detectors 104. By selectively actuating one of the emitters 102 and one of the detectors 104, the signal produced by the detector may be associated with a region of the user's neurocranium that subtends the emitter and detector. If the emitter 102 and detector 104 are disposed proximate each other, the infrared radiation that reaches the detector will have primarily passed through the superficial scalp and bone tissues and is unlikely to have penetrated brain tissues underlying the bone of the neurocranium. When the emitter 102 and detector 104 are disposed spaced further apart, the infrared radiation that reaches the detector will generally have penetrated the scalp and bone tissues and entered the underlying brain tissue.

In this embodiment, the headset 108 is in wireless communication with a controller 110, which in this embodiment is implemented using a tablet computing device acting as a host controller. The host controller is thus remotely disposed with respect to the emitters 102 and detectors 104, and the headset 108 includes a transmitter (not shown) operable to transmit the signals to the controller 110 for processing. The controller 110 receives the signals generated by the detectors 104, which are processed to determine changes in blood oxygenation within the brain tissue along the path between the respective emitter and detector. Based on the processing of the signals, the controller 110 is able to generate a spatial representation of brain activity within in the user's brain.

In one embodiment each emitter 102 is configured to produce near infrared radiation at two or more wavelengths, which are selected to cause an associated detector to produce signals that facilitate determination of a blood oxygenation state of the underlying brain tissue. For example, first and second wavelengths may be selected that fall on either side of the isobestic point for oxygenation and deoxygenation of blood hemoglobin (Hb) at which deoxy-Hb and oxy-Hb have substantially identical absorption coefficients. For example an emitter that produces wavelengths of 750 nm and 850 nm may be used. In other embodiments the selected wavelengths may fall on the same side of the isobestic point.

The detected intensity of each of the selected wavelengths at the detector 104 is thus indicative of the blood oxygenation state, which in turn is indicative of local cerebral hemodynamics within the brain tissue and facilitates a determination of neural activity within the portion of the user's brain through which the near infrared radiation produced by the emitter has traveled to reach the detector.

In this embodiment the emitters 102 and detectors 104 are disposed on the headset 108 and the controller 110 acts as a host controller, which is remotely disposed with respect to the headset and receives signals transmitted by a transmitter (not shown) on the headset 108. In this embodiment, the headset 108 may further include a headset controller (not shown) disposed on the headset and operably configured to control signal generation and acquisition by the emitters 102 and the detectors 104 and the transmission of these signals to the host controller 110.

### Emitters and Detectors

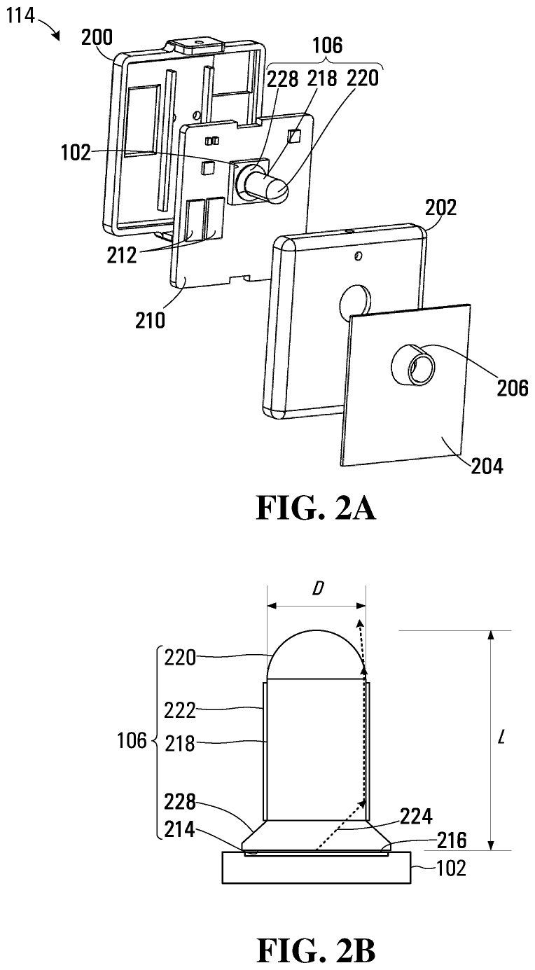

In this embodiment the detectors 104 are mounted within a detector enclosure 112, which also houses some of the emitters 102. The remaining emitters are each housed in a separate emitter enclosure 114. One of the emitter enclosures 114 is shown in exploded view in FIG. 2A. In this embodiment the emitter enclosure 114 is fabricated in two parts including a rear portion 200 and a front portion 202. The emitter enclosure 114 also includes a cover 204 having a flanged opening 206. In other embodiments the detector enclosure 112 may be otherwise configured. The emitter 102 is mounted on a circuit substrate 210 including driver circuitry 212 for driving the emitter. In one embodiment the emitter 102 may be implemented using a light source operably configured to produce the near infrared radiation at each of the first and second wavelengths. Light emitting diode packages having more than one light emitting diode operating at different wavelengths are available from several manufacturers such as Osram Sylvania of Wilmingdon Mass., USA.

Referring to FIG. 2B, the emitter 102 and light pipe 106 are shown in elevational view. The light pipe 106 has a coupling surface 214 for coupling the near infrared radiation produced by the emitter 102 into the light pipe. In the embodiment shown the coupling surface 214 is in direct contact with a window covering the radiating surface 216 of the light source(s) of the emitter 102 and the near infrared radiation is coupled directly into the light pipe 106. Minor Fresnel losses may be introduced at an optical interface between the emitter 102 and the coupling surface 214, however direct contact between the coupling surface and the emitter 102 eliminates the need for adhesives. In the configuration shown, transmission efficiency through the light pipes 106 has been found to be about 76%. The light pipe 106 further includes a guide portion 218 and a distal lens 220. In this embodiment the lens 220 is configured as a plano-convex lens, but in other embodiments the lens may have other configurations. The guide portion 218 extends between the coupling surface 214 and the distal lens 220 for guiding the near infrared radiation through the light pipe 106. The distal lens 220 is operably configured to contact the scalp of the user and direct near infrared radiation from the light pipe 106 through the scalp into the neurocranium of the user.

In the embodiment shown the light pipe 106 also includes a sheath 222 surrounding at least a portion of the guide portion 218 of the light pipe. The sheath 222 is optically absorbent at the wavelengths emitted by the emitter 102 and reduces near infrared radiation leakage from the guide portion 218 of the light pipe 106. In some embodiments the sheath 222 may include an outer surface that is ribbed or otherwise configured to divert the user's hair away from the distal lens 220 when the light pipe 106 is in contact with the scalp, thereby improving near infrared radiation coupling between the emitters 102 and the scalp.

In the embodiment shown the guide portion 218 has a generally cylindrical shape and has a diameter D selected to cause total internal reflection of near infrared radiation or light rays incident at inner surfaces of the guide portion. In one embodiment the light pipe 106 is molded from a liquid silicone rubber material (such as Lumisil LR 7601/70), which has high optical transmissivity at near infrared radiation wavelengths. Lumisil LR 7601/70 has a refractive index of 1.41, for which the total internal reflection (TIR) critical angle is about 45.2°. A light ray 224 emitted from the center of the radiating surface 216 of the emitter 102 would thus impinge on the outer surface of the guide portion 218 and would be reflected to travel along the outer surface of the guide. Any light rays from the emitter 102 at an angle greater than 45.2° that impinge on the outer surface of the guide portion 218 would escape from the light pipe 106, but would be absorbed in the sheath 222.

In the embodiment shown a cross sectional area of the guide portion 218 is smaller than a cross sectional area of the coupling surface 214 and the light pipe includes a tapered transition 228 between the coupling surface and the guide portion. A taper angle of the tapered portion 228 is selected to prevent near infrared radiation leakage from the tapered transition. In FIG. 2B, the light ray 224 at the TIR critical angle first impinges on the outer surface of the guide portion 218 well into the guide portion and the tapered portion 228 thus has negligible effect on near infrared radiation coupling into and guiding through the light pipe 106. The tapered transition 228 however provides for convenient mounting of the light pipe 106 to the emitter 102 without the use of adhesives. In the embodiment shown in FIG. 2A, the flanged opening 206 in the cover 204 has a diameter sized to correspond to the diameter D of the guide portion 218. The guide portion 218 is thus sized to permit the guide portion 218 to protrude through the opening flanged opening 206, while retaining and urging the tapered portion 228 into close contact with the radiating surface 216 of the emitter 102. The light pipe 106 may have a length of between about 7 mm and about 15 mm in one embodiment. In one embodiment, a length of at least about 7 mm (millimeters) of the light pipe 106 protrudes outwardly from a surface of the headset 108 at the flanged opening 206 in the cover 204.

In one embodiment the guide portion 218 of the light pipe 106 has a diameter of about 4 mm, the tapered portion 228 has a diameter of about 6 mm at the coupling surface 214, and the light pipe has an overall length L of about 9 mm. The plurality of emitters 102 may each be configured generally as shown in FIG. 2A and 2B.

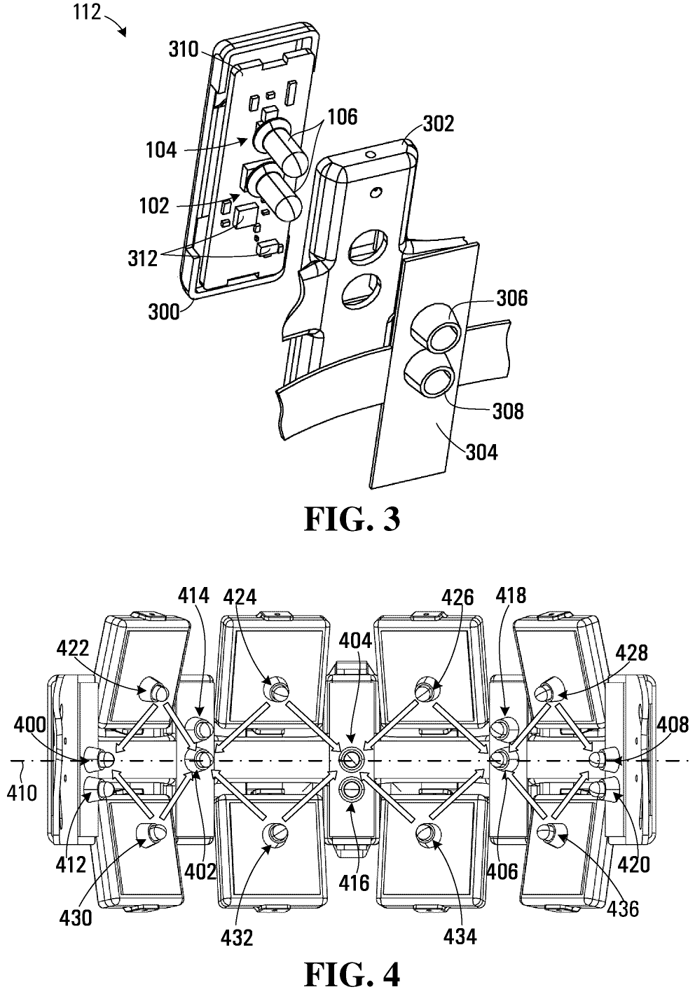

One of the detector enclosures 112 is shown in exploded view in FIG. 3. Referring to FIG. 3, the detector enclosure 112 is fabricated in two parts including a rear portion 300 and a front portion 302. The rear portion 300 and front portion are fabricated as part of a unitary assembly that will be described in more detail below. The emitter enclosure 114 also includes a cover 304 having a pair of flanged openings 306 and 308. The detector 104 and emitter 102 are both mounted on a circuit substrate 310, which includes circuitry 312 for monitoring the detector.

In this embodiment the emitter 102 is disposed proximate the detector 104 for use as a shallow path emitter. The configuration of the shallow path emitter 102 is generally similar to the configuration described above in connection with FIGS. 2A and 2B. The detector 104 may be implemented using a photodiode that is responsive to the selected emitter wavelengths. An example of a suitable photodiode is the S9674 Silicon photodiode available from Hamamatsu Photonics K.K., Japan, which has high responsivity in the wavelengths region around 800 nm and a 2 mm×2 mm photosensitive near infrared radiation receiving area. For the detector 104, the light pipe 106 may be configured substantially as shown in FIG. 2A, except that the distal lens 220 captures near infrared radiation at the point of contact with the user's scalp and directs the radiation through the light pipe to the coupling surface 214 for illuminating the detector photosensitive area. The coupling surface 214 of the light pipe 106 may directly contact the near infrared radiation receiving surface of the photodetector, as in the case of the emitters 102.

As disclosed above, in one embodiment the light pipe 106 is molded from a liquid silicone rubber material such as Lumisil LR 7601/70, which is a relatively compliant material having a Shore A durometer of 70. The material is biocompatible for skin contact for a period of time that the headset would usually be worn by a user during a brain activity assessment session. The material also has good resistance to environmental and other contaminants. The low durometer of the light pipes 106 facilitates comfortable optical contact with the scalp of the user. The inventors have found that a light pipe material having a durometer in a range of between about Shore A durometer 45 and about Shore A durometer 70 provides an acceptable level of comfort and transmittance of near infrared radiation. However, in some embodiments even more compliant materials having Shore A durometer as low as 30 may be used. Less compressible materials having Shore A durometer as high as 95 may also provide comfort for the user.

The headset 108 of FIG. 1 is shown in a view in FIG. 4 in which all of the emitters 102 and detectors 102 are visible. Referring to FIG. 4, five of the detectors 104 (numbered 400-408 in FIG. 4) are aligned along a centrally disposed band that extends laterally over and conforms to the user's crown such that the detectors are aligned along a frontal plane 410 (extending into the page) through the user's neurocranium when the headset is worn by the user. Of the five detectors 400-408, detector 404 is medially disposed while the remaining detectors are laterally spaced apart from the medial detector.

The emitters 102 include shallow path emitters (labeled as 412-420 in FIG. 4) disposed proximate to each one of the detectors 400-408. Four emitters 422-428 are disposed spaced rearwardly with respect to the frontal plane 410 and laterally offset from the detectors 400-408 such that each of the emitters is aligned between two of the detectors. Four emitters 430-436 are disposed spaced forwardly with respect to the frontal plane 410 and also laterally offset from the detectors 400-408. Each of the emitters 412-436 and the detectors 400-408 may be independently and sequentially actuated. As such, the emitters and detectors may be actuated in sequence such that a single emitter 102 is actuated to couple near infrared radiation through the scalp while a single detector 104 is simultaneously monitored to receive the near infrared radiation after traveling through the underlying brain tissue. In this embodiment, since there are only five detectors and thirteen emitters, a detector may be paired with different emitters to receive signals traveling along different paths through the underlying brain tissues. For example, the detector 402 may be sequentially paired with the emitters 414, 422, 424, 430, and 432. In other embodiments, more than one pair of emitter/detector pairs may be activated at the same time if the pairs are spaced apart or modulated at different frequencies to avoid signal interference. Additionally more than one of the shallow path emitters may be activated together with their associated proximate detectors, since the shallow path of the near infrared radiation is less likely to cause interference between signals produced by different emitter/detector pairs.

Each of the shallow path emitters 412-420 is disposed proximate to respective detectors 400-408. In one embodiment the spacing between the shallow path emitters 412-420 and the respective detectors 400-408 is about 8 mm center-to-center. Near infrared radiation that reaches the detector will have traveled over a relatively shallow path through superficial scalp and bone tissues and is unlikely to have penetrated brain tissues underlying the bone of the neurocranium. Accordingly, when one of the shallow path emitters is activated, the adjacent detector produces a shallow path signal. The emitters 422-436 are spaced apart by about 30 mm center-to-center from the nearest detector and near infrared radiation emitted by these emitters thus travels over a deeper path through the underlying brain tissues before reaching the nearest detector. In one embodiment the penetration of the near infrared radiation from the emitters 422-436 is about 15 mm into the underlying brain tissues.

In one embodiment the shallow path signals are used as a basis for filtering noise from the signals produced between the emitters 422-436 and the respective nearest detectors. Noise may be induced by blood flow in the skin, blood flow in the neurocranium, the user's cardiac pulse, movement between the headset 108 and the users scalp, and ambient light, for example. The controller 110 may be operably configured to process the shallow path signals to determine shallow path noise and make corrections to the deep path signals when determining changes in blood oxygenation within the underlying brain tissues.

### Headset

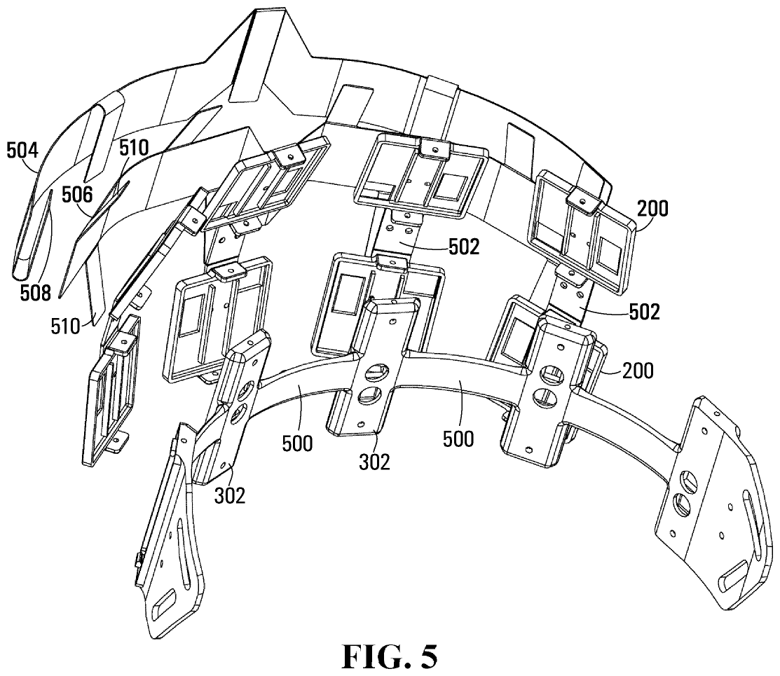

The detector enclosures 112 and emitter enclosures 114 of the headset 108 act as a plurality of articulated segments which are urged toward the scalp of the user to cause contact between the associated light pipes 106 of the respective emitters 102 or detectors 104 and the scalp. Portions of the headset 108 that operate to urge the detector enclosures 112 toward the user's scalp are shown in exploded view in FIG. 5. Referring to FIG. 5, the headset 108 includes a sprung band 500 that extends between the respective front portions 302 of adjacent detector enclosures 112. The detector enclosures 112 are thus disposed spaced apart along the sprung band 500, which has a curvature operable to conform to a corresponding lateral curvature of the user's scalp. The sprung band 500 urges the plurality of detectors 104 and the shallow path emitters 102 toward the user's scalp when the band is worn by the user, causing the associated light pipes 106 to contact the scalp.

The rear portions 200 of the emitter enclosures 114 are shown disposed in pairs, one of the pair being disposed forwardly with respect to the sprung band 500 and the other being disposed rearwardly with respect to the sprung band. Each of the rear portions 200 of the pair of emitter enclosures 114 has a spring 502 that joins between the rear portions and urges them toward the scalp when the headset 108 is worn by the user. The spring 502 causes the rear portions 200 and thus emitter enclosures 114 in each pair to be toed in to conform to a curvature of the user's neurocranium in a direction aligned with the sagittal plane.

As shown in FIG. 2A and FIG. 3, the emitters 102 and detectors 104 are mounted on the circuit substrates 210 and 310. Still referring to FIG. 5, in this embodiment the headset 108 also includes a detector flexible interconnect 504 and an emitter flexible interconnect 506. The detector flexible interconnect 504 includes flexible tabs 508 that connect to each detector circuit substrate 310 within the detector enclosures 112. Similarly, the emitter flexible interconnect 506 emitter flexible interconnect 506 includes flexible tabs 510 that connect to each emitter circuit substrate 210 within the emitter enclosures 114. In one embodiment, the flexible interconnect and the plurality of circuit substrates may be formed as a unitary flexible circuit substrate.

### Electrical and Control Systems

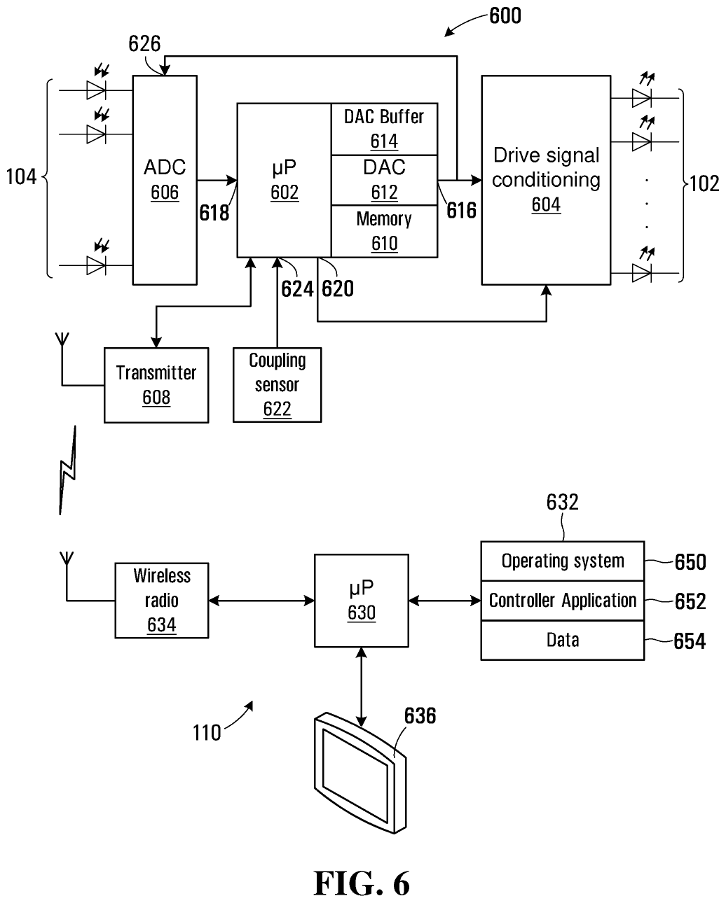

A block diagram of the electrical and control components of the apparatus 100 is shown in FIG. 6.

Referring to FIG. 6 the headset controller is shown at 600 and includes a microcontroller 602. In one embodiment the microcontroller 602 may be implemented using a ST Microelectronics controller such as the STM32F407VGT6 controller, which includes an on-board flash memory 610, digital to analog converter (DAC) 612, and a DAC buffer 614. The memory 610 includes storage for instructions for directing the microcontroller to implement headset controller functionality and storage for other data generated. The DAC buffer 614 may be part of the memory 610 and is used for storing data defining a modulation waveform for driving the emitters 102. The digital to analog converter 612 reads the data in the DAC buffer 614 and converts the digital waveform data into an emitter analog drive signal at an output 616. The headset controller 600 further includes a drive signal conditioning block 604 that conditions and directs the analog drive signal to the various emitters 102 via the driver circuitry 212 and 312 shown in FIGS. 2A and 3. The drive signal conditioning block 604 is controlled by a signal generated at an I/O output 620 of the microcontroller 602, which facilitates selection of a particular emitter or emitters to be driven at any given time. A drive signal level provided to each individual emitter 102 is set by the microcontroller 602 via a scaling factor applied to the modulation waveform for driving the emitter.

The headset controller 600 also includes an analog to digital converter (ADC) 606. Signals produced at each detector 104 are amplified and conditioned by the circuitry 312 shown in FIG. 3 prior to conversion into digital data by the analog to digital converter 606. In the embodiment shown, the output 616 of the digital to analog converter 612 is received by the analog to digital converter 606 at an input 626 for synchronous demodulation of the detector signals. The microcontroller 602 includes an input 618 for receiving data from the analog to digital converter 606. In one embodiment the input 618 may be implemented as a Serial Peripheral Interface (SPI), which is capable of providing high bandwidth data transfer from the analog to digital converter 606.

The headset controller 600 also includes a transmitter 608. The transmitter 608 may be implemented as a Bluetooth wireless interface having a relatively low power consumption which permits the headset controller 600 to be run on battery power (not shown).

In this embodiment the headset controller 600 further includes a coupling sensor 622 in communication with an I/O input 624 of the microcontroller 602 for generating a coupling signal indicating a state of coupling between the plurality of light pipes of the emitters 102 and detectors 104 and the user's scalp. The coupling sensor 622 may be a capacitive sensor disposed on the headset 108 that produces a signal indicative of the proximity of the headset 108 to the scalp of the user. A reduction in sensed capacitance would be indicative of the headset 108 having been moved or removed such that the emitters 102 and detectors 104 are no longer in contact with the user's scalp. Alternatively or additionally, an acoustic sensor such as a microphone may be disposed on the headset 108 to generate a signal that in response to an ambient sound level at the microphone. An increase in sound level at the microphone may indicate that the headset 108 has been moved or removed. In other embodiments an accelerometer may be disposed on the headset 108 to provide inertial signals indicative of movement of the headset. Rapid movements of the headset 108 sensed by the accelerometer may be indicative that the headset 108 has been moved or removed. Another alternative would be to monitor ambient light signals experienced at one or more of the detectors 104. When an ambient light component in the detector signal changes significantly, this may be indicative of the headset 108 being removed from the user's scalp. In one embodiment two or more of the alternative coupling sensors may be implemented to monitor the coupling conditions between the headset 108 and the user's scalp.

In this embodiment the headset controller 600 is in communication with the host controller 110, which includes a microprocessor 630, a memory 632, a wireless radio 634, and a display 636. In the embodiment shown in FIG. 1, the host controller 110 is shown as a tablet computing device in which the display 636 is implemented as a touch sensitive screen for receiving user input. However in other embodiments the host controller 110 may be implemented using a conventional computing device such as a laptop or desktop computer or other computing device. The memory 632 includes storage 650 for storing codes that direct the microprocessor 630 to provide functions for implementing an operating system, such as an Android OS, iOS, Windows or other operating system. The memory 632 also includes storage 652 for storing codes that direct the microprocessor 630 to perform controller functions for interacting with and controlling the headset 108. The memory 632 further includes storage 654 storing data associated with performing brain activity monitoring.

In one embodiment the processor circuit 630 may include a graphic processing unit operably configured to accelerate processing of the signals from each of the plurality of detectors 104 to facilitate near real time presentation of results to the user.

The wireless radio 634 implements Bluetooth communication protocols for communicating with the transmitter 608 of the headset controller 600. In other embodiments the wireless radio 634 may implement other wireless protocols for communicating with the transmitter 608, which may be correspondingly configured to implement a wireless protocol other than the Bluetooth protocol.

### Headset Controller Process

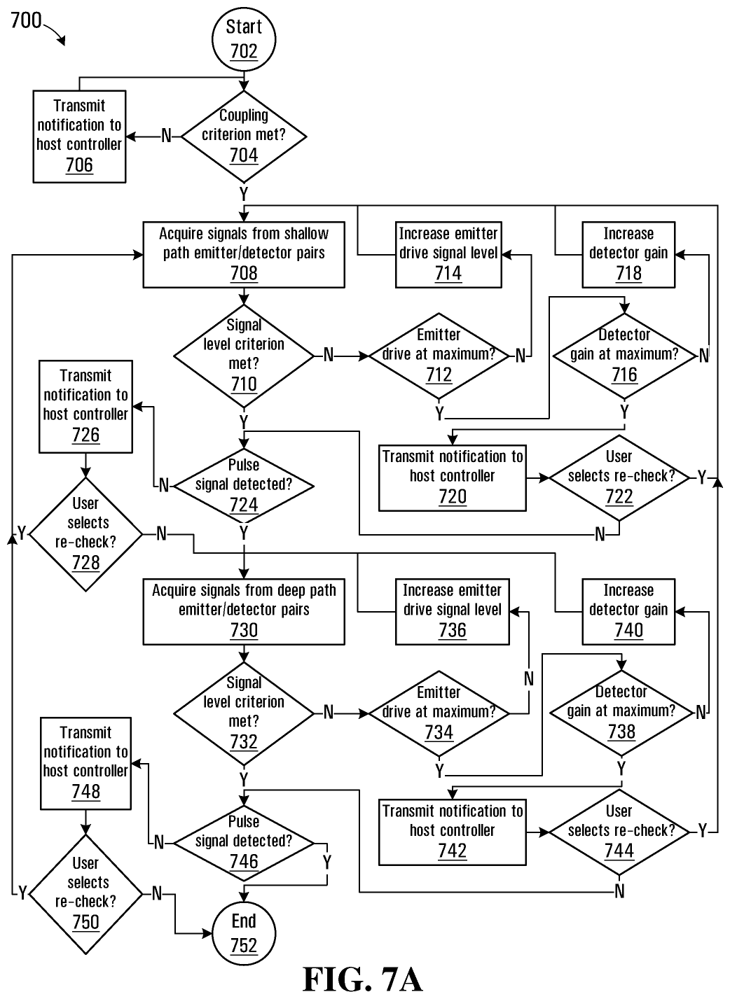

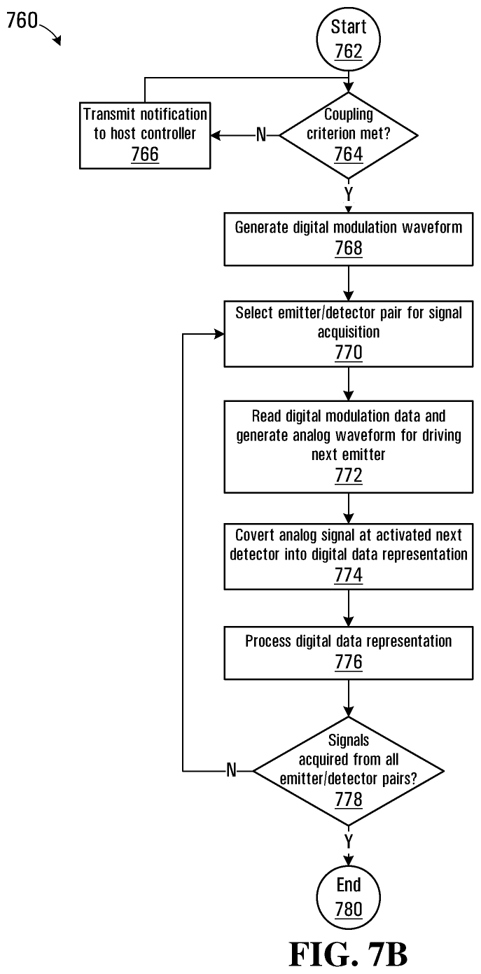

Referring to FIG. 7A, a flowchart depicting blocks of code for directing the headset controller 600 to perform a signal calibration is shown generally at 700. The blocks generally represent codes that may be read from the memory 610 for directing the microcontroller 602 to perform signal acquisition. The actual code to implement each block may be written in any suitable program language, such as C, C++, C#, Java, and/or assembly code, for example.

The signal calibration process 700 begins at block 702 when user initiates a signal calibration at the host controller 110. Block 704 directs the microcontroller 602 to determine whether a coupling criterion has been met by reading the coupling sensor 622 and comparing the coupling signal received at the I/O input 624 against a range of values determined to indicate that the coupling to the user's scalp is sufficient. If at block 704, the coupling criterion is not met then the microcontroller 602 is directed to block 706, which directs the microcontroller to transmit a notification to the host controller 110 for display to the user. The user may then relocate the headset on the scalp in an attempt to improve the coupling. Block 706 then directs the microprocessor 602 back to block 704 to re-check the coupling. When at block 704, the coupling criterion is met, the microcontroller 602 is directed to block 708. Block 708 directs the microcontroller 602 to acquire signals from the shallow path emitters 412-420 and the associated detectors. The signal acquisition process for emitter/detector pairs is shown in FIG. 7B and is described in more detail later herein.

The signal calibration process 700 then continues at block 710, which directs the microcontroller 602 to determine whether the signal level produced by each detector for each respective emitter/detector pair falls within a pre-determined signal level criterion. If the signal level criterion is not met at block 710, the microcontroller 602 is directed to block 712, which directs the microcontroller to determine whether the emitter signal level is at a maximum. If the signal level not yet maximized, block 712 directs the microcontroller 602 to block 714, which directs the microcontroller to increase the emitter drive signal level for the emitter. The process then continues by repeating block 708 and 710. If at block 712, the signal level is already maximized, the microcontroller 602 is directed to block 716, which directs the microcontroller to determine whether the detector gain is already at a maximum. If the detector gain has not already been maximized then block 716 directs the microcontroller 602 to block 718 and the gain of the detector is increased. Block 718 may also reduce the emitter drive signal level back to a lower level or a minimum level. Block 718 then directs the microcontroller 602 back to block 708 and blocks 708 and 710 are repeated with the increased detector gain.

If at block 716 the detector gain is at a maximum, the microcontroller 602 is directed to block 720. Block 720 directs the microcontroller to transmit a notification message to the host controller 110 causing a message to be displayed for the user on the host controller. The user may adjust the headset position and elect to re-check, in which case block 722 directs the microcontroller 602 back to block 708 to repeat signal acquisition of the shallow path signals for the adjusted headset position. Alternatively, the user may elect to continue with the headset coupling as-is, in which case block 722 directs the microcontroller 602 to block 724.

Block 724 then directs the microcontroller 602 to determine whether a cardiac pulse signal has been detected. The microcontroller 602 is directed to extract a cardiac pulse signal from the signal received at the detector, which is relatively strong compared to other signal components and also has a well-known waveform that facilitates extraction. If at block 724 the cardiac pulse signal is not detected, the microcontroller 602 is directed to block 726, which directs the microcontroller to transmit a notification message to the host controller 110. The user may then adjust the headset position and elect to re-check, in which case block 728 directs the microcontroller 602 back to block 708 to repeat the shallow path signal acquisition for the new headset position. Alternatively, the user may elect to continue with the coupling as-is, in which case block 728 directs the microcontroller 602 to block 730.

If at block 724 the cardiac pulse signal is detected in the shallow path signal, the microcontroller 602 is directed to block 730. The cardiac pulse signal provides an additional determination of the effectiveness of the coupling between the headset 108 and the user's scalp and is further used to perform filtering to remove physiological effects from signals not related to changes in blood oxygenation that are indicative of brain activity.

The signal calibration process 700 then continues at block 730, which directs the microcontroller 602 to repeat the process for the deep path emitter/detector pairs substantially as described above in connection with the shallow path signals. The signal levels for driving the deep path emitters 422-436 and the detector gain is thus calibrated at blocks 730-740 to bring the signals within the signal level criterion for successful detection by the associated detectors. When the emitter drive signal level and detector gain are maximized and the user has adjusted the headset position and elected to re-check at block 744, the microcontroller 602 is directed back to block 708 to repeat the signal acquisition for shallow path emitters at the new headset position.

The microcontroller 602 is also directed to determine whether the cardiac pulse signal is detected for the deep path emitter/detector pairs at block 746. When the pulse signal is not detected at block 746 and the user has adjusted the headset position and elected to re-check at block 750, the microcontroller 602 is directed back to block 708 to repeat the signal acquisition for shallow path emitters. If the signal level criterion is met at block 732 and the cardiac pulse is detected at block 746, the signal calibration process 700 successfully ends at block 752.

Referring to FIG. 7B, a flowchart depicting blocks of code for directing the headset controller 600 to acquire signals is shown generally at 760. The signal acquisition process 760 starts at block 762, which directs the microcontroller 602 to commence data acquisition. In one embodiment the host controller 110 initiates the monitoring activity, but in other embodiments such as the signal calibration process 700 shown in FIG. 7A the activity may be initiated at the headset 108. Blocks 764 and 766 are optionally included to direct the microcontroller 602 to determine whether the coupling criterion has been met and to alert the user if not met. The host controller 110 may be operably configured to discontinue monitoring activities when the coupling criterion is indicative of a plurality of the emitters or detectors not being coupled to the scalp of the user.

If the coupling criterion is met at block 764, block 768 then directs the microcontroller 602 to generate data representing a digital waveform for driving the emitters 102. In one embodiment the modulation waveform is a sinewave having a frequency in the kilohertz range and a duration of about 4 milliseconds. Other embodiments may implement different waveforms, frequencies, and/or duration. Block 768 also directs the microcontroller 602 to store the waveform data in the DAC buffer 614. In one embodiment the same digital waveform may be used for signal acquisition from each of the different emitter/detector pairs with a calibration scaling factor being applied to the waveform for each emitter/detector pair as determined by the signal calibration process 700.

The signal acquisition process 760 then continues at block 770, which directs the microcontroller 602 to select an emitter/detector pair that is to be activated for signal acquisition. The process 760 may be used to acquire signals from one emitter/detector pair or from a group of emitter/detector pairs, as in the case of the signal calibration process 700. Each of the emitters 102 is paired with one of the detectors 104, which as a pair define a measurement channel that can be activated. Block 770 directs the microcontroller 602 to cause the selected detector 104 to be configured for receiving signals via the analog to digital converter 606. Block 770 also directs the microcontroller 602 to configure the drive signal conditioning block 604 via the I/O signal 620 to connect the selected emitter to produce near infrared radiation. In embodiments where the emitter is operably configured to produce near infrared radiation at multiple wavelengths, the drive signal conditioning block 604 may also configure the emitter to selectively enable each of the wavelength sources in the emitter to generate the respective wavelengths.

Block 772 then directs the microcontroller 602 to cause the digital to analog converter 612 to read the digital modulation waveform data stored in the DAC buffer 614 and to commence conversion of the digital data into an analog waveform. If the signal calibration process 700 has already been performed, the microcontroller 602 would also apply any determined calibration factor for driving the emitter at a signal level that produces sufficient signal at the associated detector. The analog waveform at the output 616 is thus connected through appropriate drive signal buffers in the drive signal conditioning block 604 to the selected emitter, which then generates a frequency burst having a duration and drive level set by the digital modulation data and the determined signal level calibration factor. The selected emitter couples near infrared radiation through the scalp, which travels through the underlying tissue such that at least a portion of reaches the selected detector and produces an analog signal representing the received near infrared radiation. In embodiments where the emitter 102 includes multiple wavelength sources, each wavelength is activated separately to facilitate generation of separate signals at the detector for each wavelength.

The signal acquisition process 760 then continues at block 774, which directs the microcontroller 602 to cause the analog to digital converter 606 to convert the analog signal received at the selected detector into digital data representation, which is received at the input 618 of the microcontroller as a digital data stream. For an emitter 102 that operates at multiple wavelengths, digital data streams for each wavelength will thus be produced by the detector. Block 776 then directs the microcontroller 602 to process the digital data signals. During the signal calibration process 700 the microcontroller 602 processes the digital data representation to determine signal level and to extract a cardiac pulse signal, if present. Optionally, the processing at block 776 may further involve the microcontroller 602 causing the transmitter 608 to transmit the digital signal to the host controller 110 via the wireless Bluetooth connection for further processing by the host controller.

The process 760 then continues at block 778, which directs the microcontroller 602 to determine whether signals have been acquired for all required emitter/detector pairs. If there remain further signals to be acquired, the microcontroller 602 is directed to block 770, which directs the microcontroller to select the next emitter/detector pair for activation and blocks 772-778 are repeated for the next emitter/detector pair. If at block 778 there are no further signals to be acquired, the microcontroller 602 is directed to block 780 where the signal acquisition process ends.

Following completion of the signal calibration process 700, a user assessment session may be commenced in which signals are acquired from the various the emitter/detector pairs for the duration of the session. For example, referring back to FIG. 4, the shallow path emitter 412 may be actuated together with the detector 400 to read the shallow path signal. This may be followed by the emitter 422 being actuated together with the detector 400 and then the emitter 430 together with the detector 400. The activation sequence may then be repeated with the shallow path emitter 414 and detector 402, followed by activation of the emitters 422 and then the emitter 430 together with the detector 400. The activation sequence may then continue with emitters 424 and 432 and so on. In the emitter and detector layout shown in FIG. 4, the large arrows indicate 16 different deep path measurement channels that can be made by combining various ones of the emitters 102 with each detector 104. Additional shallow path measurement channels also exist between each of the shallow path emitters 412-420 and the detectors 104. These deep path measurement channels and shallow path measurement channels may be activated one-by-one in a sequence and various combinations of activation sequence may be implemented. In some embodiments two or more emitters and/or detectors may be simultaneously activated.

### Host Controller Process

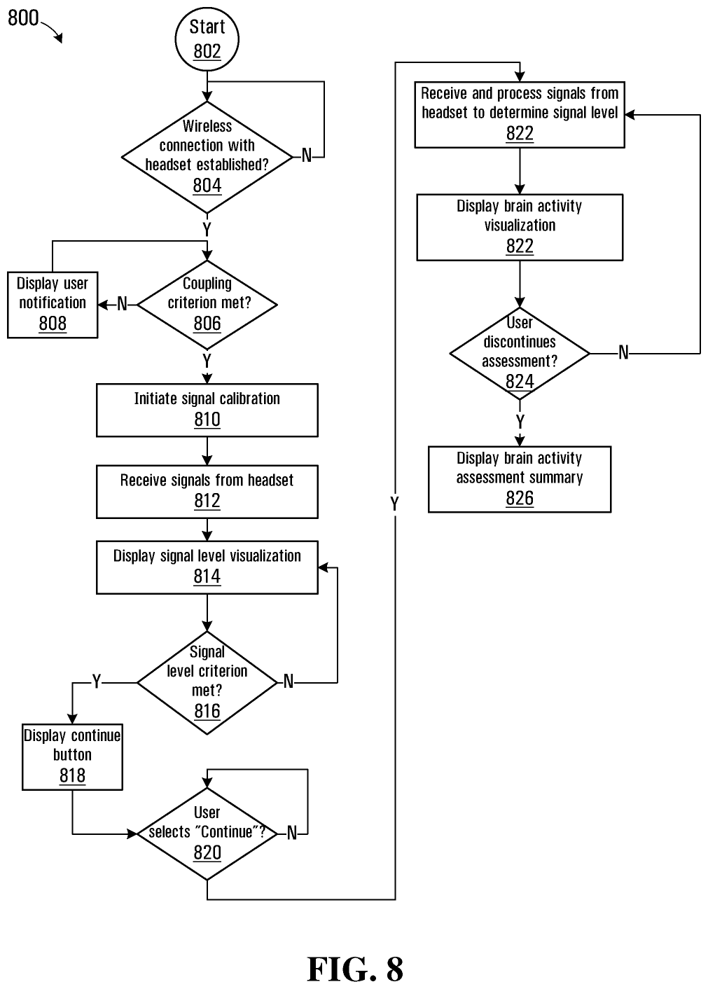

Referring to FIG. 8, a flowchart depicting blocks of code for directing the processor circuit of the host controller 110 to perform an assessment session is shown generally at 800. The blocks generally represent codes that may be read from the controller application storage 652 in the memory 632 for directing the microprocessor 630 to perform the assessment. The actual code to implement each block may be written in any suitable program language, such as C, C++, C#, Java, and/or assembly code, for example.

A brain activity assessment commences at block 802 when a user launches the application and the microprocessor 630 is directed to execute the codes stored in the storage location 652 of the memory 632. The application may initially go through a process of receiving user details that will be associated with the assessment session. Block 804 then directs the microprocessor 630 to attempt to establish a wireless connection between the wireless radio 634 of the host controller 110 and the transmitter 608 on the headset 108. If at block 804 no wireless connection is established, the microprocessor 630 is directed to repeat block 804.

If at block 804 a wireless connection with the headset 108 is established, the microprocessor 630 is directed to block 806. Block 806 directs the microprocessor 630 to determine whether the coupling criterion for the headset 108 has been met. As described above in connection with the signal calibration process 700 and signal acquisition process 760, when it is determined by the headset controller 600 that the headset 108 has been removed or moved on the user's scalp such that the coupling criterion is no longer met, a message is transmitted to the host controller 110. Block 806 thus directs the microprocessor 630 to determine whether the coupling criterion is currently being met at the headset 108. If the coupling criterion is not being met, block 808 directs the microprocessor 630 to cause a user notification (not shown) to be generated and displayed on the display 636 to prompt the user to put on or relocate the headset.

If at block 806 the coupling criterion is being met, block 806 directs the microprocessor 630 to block 810, which directs the microprocessor to transmit an instruction via the wireless radio 634 to the headset controller 600 to initiate the signal calibration process 700 shown in FIG. 7A. The assessment process 800 then continues at block 812, which directs the microprocessor 630 to receive the digital signal representations generated by the detectors 104 on the headset 108 during the signal calibration process 700 and transmitted via the transmitter 608 to the host controller 110. Block 812 also directs the microprocessor 630 to determine a signal level associated with each received signal.

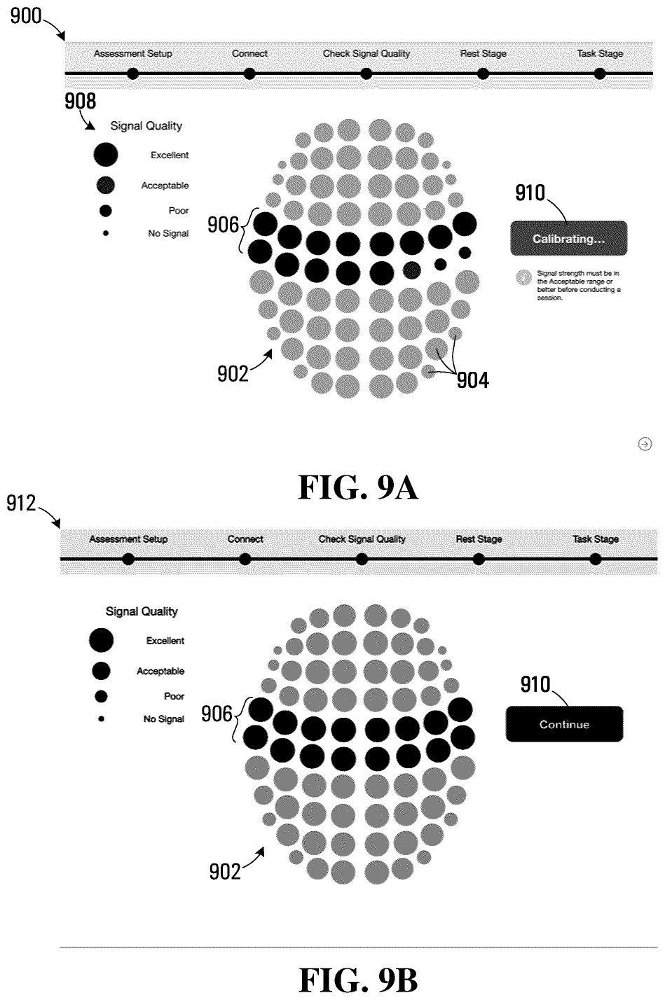

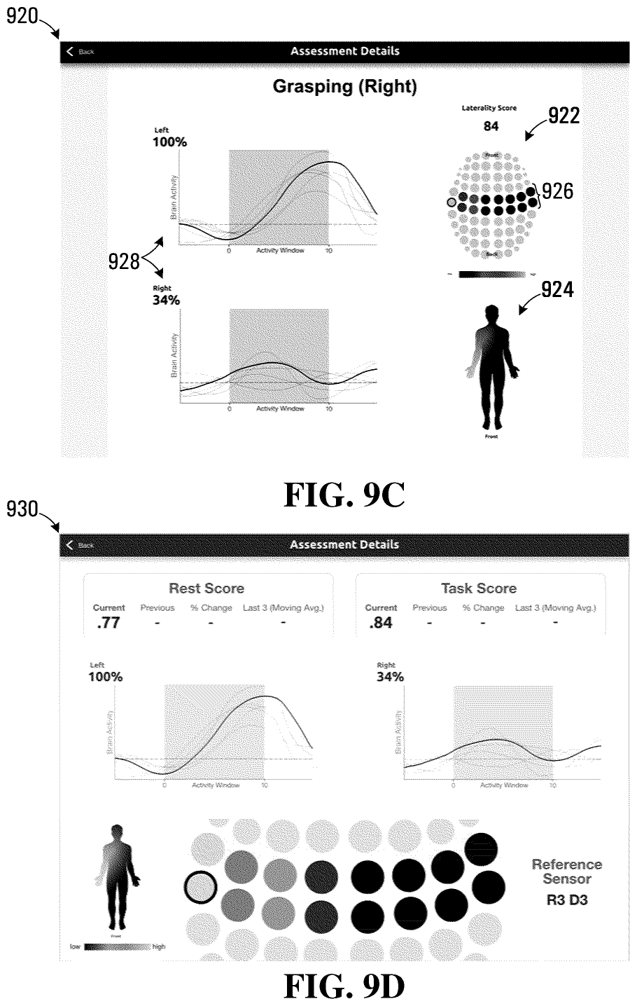

Block 814 then directs the microprocessor 630 to generate data to cause a graphical depiction of the signal quality to be displayed on the display 636 of the host controller 110 to provide feedback to the user for properly locating the headset 108 on the user's scalp. Referring to FIG. 9A, a first screenshot of the graphical depiction during signal quality evaluation is shown at 900 and includes a brain representation 902 including a plurality of dots 904 that act as a spatial representation of the deep path emitter/detector pairs along with display information indicating whether the signal intensity is within the detection range of the associated detector. An assessment region is depicted by coloring a subset 906 of the plurality of dots 904 (shown as 16 dark shaded regions in FIG. 9A). Each dot 906 represents one of the signals generated by the headset 108 for the 16 different deep path signal acquisitions represented in FIG. 4 by the large arrows. As shown in FIG. 9, some of the dots 906 have a smaller diameter than others. The diameter of each dot 906 is used as an indicator of signal level or quality for the associated path in accordance with a key 908 displayed alongside the brain representation 902. The larger two dot sizes in the key 908 are shown to indicate “Acceptable” and “Excellent” signal quality, while the smaller dot sizes are associated with “poor” signal quality or “no signal”.

Referring back to FIG. 8, the process 800 then continues at block 816, which directs the microprocessor 630 to determine whether a signal level criterion is currently being met. If some of the signal levels are not at or above the “Acceptable” level, the signal level criterion is currently not being met and the microprocessor 630 is directed to block 814. The headset controller 600 thus monitors the signal level produced at each detector and to control a level of near infrared radiation produced by the selectively actuated emitter and attempts to maintain the signal intensity within a detection range of the detector.

While at block 816 the signal level criterion is currently not being met, the graphical depiction 900 includes a status indicator 910 “Calibrating”, which indicates that the signal quality is still being evaluated. If at block 816 the signal level criterion is currently being met, the microprocessor 630 is directed to block 818, which directs the microprocessor 630 to change the displayed screen to the state shown in FIG. 9B at 912, where the status indicator 910 changes to “Continue” and all of the dots 906 in the brain representation 902 are shown in the “Acceptable” range or in the case of FIG. 9B, in the “Excellent” range. The status indicator 910 thus prompts the user to continue with the assessment session.

The assessment process 800 then continues at block 820, which directs the microprocessor 630 to wait until the user has activated the “Continue” status indicator 910 to continue with the assessment. When the user activates the “Continue” status indicator 910, block 820 directs the microprocessor 630 to block 822. Block 822 directs the microprocessor 630 to continue to receive the digital signal representations from the headset 108 and to process the signals to determine results for the assessment. Generally the signals received at the detectors will have significant noise and may also have significant components due to physiological processes such as the cardiac pulse that may obscure blood oxygenation information in the signals. The processing of the signals is described in more detail later herein.

Block 824 then directs the microprocessor 630 to generate and display results of the assessment. In one embodiment the microprocessor 630 causes a result screen shown in FIG. 9 at 920 to be displayed while the assessment is in progress. The result screen 920 shows a brain representation 922. Dots 926 associated with the various deep path signals are colored (shown as shaded in FIG. 9C) to indicate a level of brain activity. The result screen 920 also includes a corresponding body representation 924, in which regions of the body that are being activated are shaded to correspond to portions of the motor cortex of the brain that are associated with movement of those of the regions of the body. In this case the activity is a grasping action of the right hand. Various other portions of the body may be activated and the associated brain activity recorded while the results are displayed for the user as in the example shown in FIG. 9C. The graphs 928 show changes in blood oxygenation over measured during an assessment. Each assessment may include series of trials of an exercise each being timed to have a period of rest followed by a period of activity (in this particular case 10 seconds of activity). The graphs 928 depict a user's brain activity during these exercises. The thin lines each represent multiple trials for an exercise over the 10 second activity window, with periods of rest outside the activity window. The thick line represents a mean of the multiple trials.

Referring back to FIG. 8, the assessment process 800 then continues at block 824, which directs the microprocessor 630 to determine whether the user has discontinued the assessment session. If at block 824 the assessment session is not determined to have been discontinued, the microprocessor 630 is directed back to block 822 to continue receiving and processing signals. In one embodiment, block 824 further directs the microprocessor 630 to determine whether a message has been received at the host controller 110 indicating that the headset 108 has been removed or moved on the user's scalp such that the coupling criterion is no longer met. If at block 768 of the signal acquisition process 760, a notification message is transmitted by the headset controller 600 that the coupling criteria is not being met, then the microprocessor 630 determines that the assessment should be discontinued.

If at block 824 the assessment session is determined to have been discontinued, the microprocessor 630 is directed to block 826, where the microprocessor is directed to display a brain activity result summary. An example of the summary is shown in FIG. 9D at 930 and includes assessment details for both right and left grasping activities along with other metrics for a selected measurement channel (in this case the R3D3 sensor indicated by the outlined circle in the summary 930.

### Signal Processing

As disclosed above, the processing of the signals at block 822 may involve steps that suppress components of the signal that do not relate to blood oxygenation changes within brain tissue. For example, physiological processes unrelated to brain activity such as the cardiac pulse, respiration, changes in blood pressure, have an effect on how the infrared radiation is absorbed by tissues through which the radiation travels between the emitters 102 and detectors 104. Signals unrelated to brain activity are herein referred to as “contamination signals”.

As disclosed above in connection with FIG. 4, the headset 108 implements multiple channels including deep path measurement channels where the emitter and detector are separated by a sufficient distance for infrared radiation to pass through brain tissues underlying the scalp before reaching the detector. The deep path measurement channels will thus generate deep path signals including signal components associated with blood oxygenation in the brain along with other components related to physiological processes (i.e. the contamination signals). The headset 108 also implements shallow path measurement channels where the emitter and detector are separated by a distance that is too short for the infrared radiation pass through brain tissue before reaching the detector. For the shallow path measurement channels the infrared radiation from the emitter does not reach the brain tissue and the resulting shallow path signals thus do not include components associated with blood oxygenation in the brain tissues. These shallow path signals thus provide a useful representation of the contamination signals and may be used to remove or filter contamination signal components from the deep path signals. The filtered deep path signals will have components related to blood oxygenation changes within brain tissue enhanced while contamination signal components are diminished.

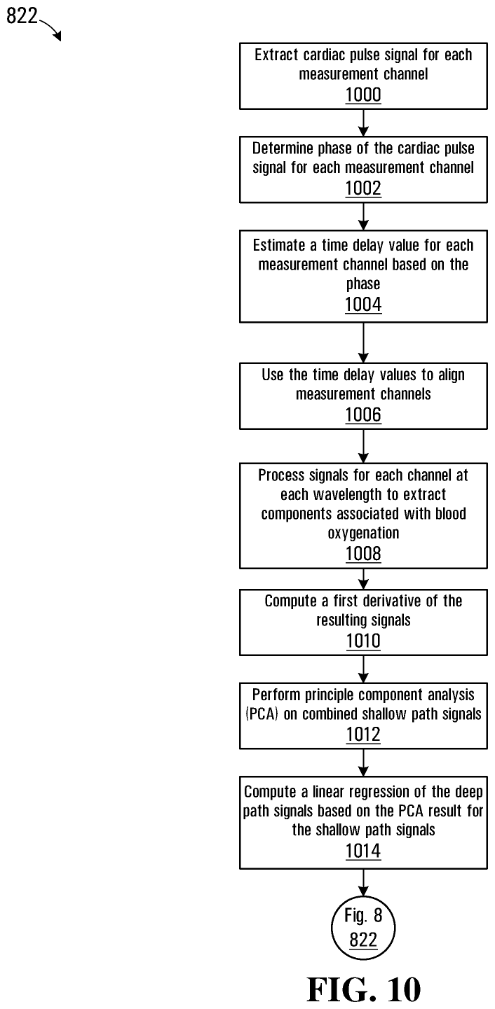

There are some difficulties in performing the filtering of the deep path signals in that the physical processes and structures of the circulatory system may cause a variable delay between contamination signals generated for different deep path and shallow path measurement channels due to these channels being spaced apart about and within the user's neurocranium. There may also be differences between how the physiological process manifest for different deep path and shallow path measurement channels. A flowchart including blocks of code for directing the microprocessor 630 of the host controller 110 to implement block 822 of the process 800 is shown in FIG. 10. Referring to FIG. 10, the process 822 begins at block 1000, which directs the microprocessor 630 to extract a cardiac pulse signal from the signal associated with each of the shallow path and deep path measurement channels. The cardiac pulse signal component is relatively strong compared to other signal components and also has a well-known waveform which facilitates extraction from the shallow path and deep path measurement channels. In other embodiments cyclic signals associated with other physiological processes such as respiration or blood pressure cyclic changes such as Mayer waves.

Block 1002 then directs the microprocessor 630 to determine the relative phase of each of the extracted cardiac pulse signals using one of the channels as a reference channel. Block 1004 then directs the microprocessor 630 to estimate a time delay for each channel relative to the reference channel (i.e. the reference channel is assumed to have zero time delay). The process 822 then continues at block 1006, which directs the microprocessor 630 to align the deep path and shallow path signals for all the measurement channels. The processed signals at block 1006 thus have the variable delays due to the manifestation of the physiological processes on the respective signals all aligned so that the contamination signals are substantially aligned in time for all measurement channels.

In some embodiments, the absence of a cardiac pulse signal in detected signals may be taken as being indicative that the coupling criterion is no longer being met. The headset controller 600 or the host controller 110 may be operably configured to process the signal received by at least one of the detectors to extract a pulse signal representing a detected heartbeat of the user and to monitor the pulse signal to determine whether coupling between the emitters and detectors and the scalp of the user meets a coupling criterion. A dominant frequency may be extracted from the detected signals, and if the frequency falls within a frequency range based on the user's expected heartbeat frequency range, then the coupling criterion will be considered to be met. The cardiac pulse signal may thus be used in addition to or instead of the coupling signal produced by the coupling sensor 622 (shown in FIG. 6).

Block 1008 then directs the microprocessor 630 to process signals for each channel and at each wavelength to extract components associated with blood oxygenation. For example, in embodiments where a dual wavelength emitter having wavelengths of 750 nm and 850 nm is used, the components for each of these wavelengths may be extracted from the signals for each channel to yield a signal that is indicative of blood oxygenation associated with the channel.

Block 1010 then directs the microprocessor 630 to compute a first derivative of the signal produced at block 1008. The inventors have found that signals that reflect a rate of change in blood oxygenation are less noisy than raw blood oxygenation signals.

The process 822 then continues at block 1012, which directs the microprocessor 630 to preform principal component analysis (PCA) on the combined shallow path signals to generate an estimate for the contamination signals. In one embodiment the principal component analysis is applied more than once to the processed shallow path signals to differentiate between faster-cycling signals (for example cardiac pulse in the 0.5 Hz-2 Hz range) and slower-cycling signals (for example respiration in the 0.01 Hz-0.1 Hz range). For example, a first principal component analysis may be performed on the shallow path signals produced at block 1010. This may be followed by a second principal component analysis on a high pass filtered version of the signals produced at block 1010 to selectively retain only fast-cycling signals. A further principal component analysis may be performed on a low pass filtered version of the signals produced at block 1010 to selectively retain only slow-cycling signals. The inventors have found that it is possible for a simple single-pass principal component analysis applied to the signals at block 1010 may capture either one of these fast or slow cycling components while possibly missing the other.

Block 1014 then directs the microprocessor 630 to compute a linear regression on the deep path signal produced at block 1010 for each deep path measurement channel. The linear regression predicts the deep path measurements as an additive function of all the contamination signals obtained by the principal component analysis, thereby estimating the influence of each contamination signal and providing a formula to then remove these influences from the deep path signals. The resulting signals have the influence of contamination signals substantially reduced to provide a signal representing changes in blood oxygenation that can be used to produce the result screen 920 shown in FIG. 9C.

Functions described above as being implemented on either the host controller 110 or the headset controller 600 may be moved between the controllers or implemented on another controller (not shown).

On other embodiments the headset controller 600 may be implemented using more than one microcontroller located on the headset 108.

While specific embodiments have been described and illustrated, such embodiments should be considered illustrative only and not as limiting the disclosed embodiments as construed in accordance with the accompanying claims.

1. An apparatus for monitoring brain activity of a user, the apparatus comprising:
a plurality of spatially separated emitters operable to generate infrared radiation;
a plurality of spatially separated infrared radiation detectors;
a plurality of light pipes urged into contact with the user's scalp, each one of the plurality of emitters and detectors having an associated light pipe operable to couple infrared radiation from the emitter into the scalp or to couple infrared radiation from the scalp to the detector;
wherein each detector is operable to produce a signal representing an intensity of infrared radiation generated by a selectively actuated one of the plurality of emitters and received at the detector after traveling on a path through underlying brain tissue, the signals being received by a controller operably configured to:
process the signals from each detector to determine changes in blood oxygenation within the brain tissue along the path between the respective emitter and detector; and
generate a spatial representation of brain activity within in the user's brain based on the processed signals.

2. The apparatus of claim 1 wherein the emitters and detectors are disposed on a headset and wherein the controller is remotely disposed with respect to the headset and wherein the headset comprises a transmitter operable to transmit the signals to the controller for processing; optionally further comprising a headset controller disposed on the headset and operably configured to control functions of the transmitter, the emitters, and the detectors.

3. (canceled)

4. The apparatus of claim 1 wherein the infrared radiation comprises near infrared radiation.

5. The apparatus of claim 1 wherein the emitter comprises a light emitting diode operably configured to produce the infrared radiation at a plurality of wavelengths selected to cause the detector to produce signals that facilitate determination of a blood oxygenation state of the brain tissue underlying each of the spatially separated emitters and associated detectors, the blood oxygenation state being indicative of local cerebral hemodynamics within the brain tissue and facilitating a determination of neural activity within the user's brain
optionally wherein the plurality of wavelengths comprises at least first and second wavelengths selected to fall on either side of the isobestic point for oxygenation and deoxygenation of blood hemoglobin;
or optionally wherein the light emitting diode associated with each of the plurality of emitters is mounted within a headset, the headset being operable to support the plurality of emitters and plurality of detectors in contact with the user's scalp when worn by the user.

6-7. (canceled)

8. The apparatus of claim 1 wherein the plurality of emitters comprises:
at least one emitter disposed proximate to one of the plurality of detectors and wherein the detector is operable to produce a shallow path signal representing an intensity of infrared radiation generated after traveling along a shallow path through scalp and bone tissue between the at least one emitter and the detector; at least one emitter disposed spaced apart from one or more of the plurality of detectors and wherein the one or more detectors are operable to produce a deep path signal representing an intensity of infrared radiation generated after traveling along a deep path through the underlying brain tissue between the at least one emitter and the one or more detectors;
optionally wherein the controller is operably configured: to activate selected emitters and detectors to generate signals associated with different paths of travel of the infrared radiation through the brain tissue; or to process the shallow path signals to determine shallow path noise, the shallow path noise being used as a basis for filtering the deep path signal to determine the changes in blood oxygenation within the brain tissue; optionally wherein the controller is operably configured to process the signals by
aligning a phase of each of the shallow path signals and deep path signals based on a physiological process component in the signals;
performing a principle component analysis on the shallow path signals to determine contamination components associated with physiological processes other than changes in blood oxygenation within the brain tissue; and
removing the contamination components from the deep path signals to provide signals representing changes in blood oxygenation within the brain tissue from which the effects of other physiological processes have been filtered;
optionally wherein performing the principle component analysis comprises:
filtering the shallow path signals to separate the shallow path signals into slow-cycling signals associated with slow-cycling physiological processes and fast-cycling signals associated with fast-cycling physiological processes; and
performing principle component analysis on each of the shallow path signals, the slow-cycling signals and the fast-cycling signals; or
wherein the controller is operably configured to, prior to performing the principle component analysis:
process the phase aligned shallow path signals to generate signals representing oxygenation and deoxygenation of blood hemoglobin; and
take a first derivative of the signals representing oxygenation and deoxygenation of blood hemoglobin.

9-13. (canceled)

14. The apparatus of claim 1 wherein each light pipe comprises a low durometer material that is optically transmissive at wavelengths associated with the infrared radiation, the low durometer material facilitating comfortable optical contact with the scalp of the user; optionally wherein the light pipe material has a durometer in a range of between about Shore A durometer 30 and about Shore A durometer 90.

15. (canceled)

16. The apparatus of claim 1 wherein (a) the length of each light pipe is between about 7 millimeters and 15 millimeters; optionally wherein each of the plurality of emitters and detectors is mounted on a headset that conforms to the scalp of the user and wherein a length of at least about 7 mm of the light pipe protrudes outwardly from a surface of the headset or
(b) each light pipe comprises:
a coupling surface for coupling infrared radiation between the light pipe and the emitter or detector;
a distal lens operably configured to contact the scalp and direct infrared radiation to or from the light pipe;
a guide portion extending between the coupling surface and the distal lens;
optionally wherein the guide portion of the light pipe has a generally cylindrical shape and has a diameter selected to cause total internal reflection of infrared radiation incident at inner surfaces of the guide portion; and optionally. wherein a cross sectional area of the guide portion is smaller than a cross sectional area of the coupling surface and the light pipe further comprises a tapered transition between the coupling surface and the guide portion and wherein a taper angle of the tapered transition is selected to prevent infrared radiation leakage from the tapered transition, the tapered transition further providing for mounting of the light pipe to the emitter or detector; and
optionally a sheath surrounding at least a portion of the guide portion of each light pipe, the sheath being operably configured to reduce infrared radiation leakage from the guide portion of the light pipe;
and optionally wherein the sheath comprises an outer surface operably configured to divert the user's hair away from the distal lens when the light pipe is in contact with the scalp.

17-21. (canceled)

22. The apparatus of claim 1 wherein the coupling surface of the light pipe is operably configured to directly contact a radiating surface of the emitter or a radiation receiving surface of the detector for coupling infrared radiation between the light pipe and the detector.

23. (canceled)

24. The apparatus of claim 1 further comprising a headset having a plurality of articulated segments, each articulated segment supporting at least one emitter or detector, the articulated segments each being urged toward the scalp of the user to cause contact between the associated light pipes of the respective emitters or detectors and the scalp;
optionally wherein each of the plurality of articulated segments is operably configured to mount a circuit substrate and wherein at least one detector or emitter is mounted on each circuit substrate;
optionally further comprising a flexible interconnect interconnecting between a headset controller and the plurality of circuit substrates;
and optionally wherein the flexible interconnect and the plurality of circuit substrates are formed as a unitary flexible circuit substrate.

25-27. (canceled)

28. The apparatus of claim 1 wherein the plurality of detectors is disposed spaced apart along a sprung band having a curvature operable to conform to a corresponding lateral curvature of the user's scalp and urge the plurality of detectors toward the scalp when the band is worn by the user; and optionally further comprising a plurality of articulated segments disposed forwardly or rearwardly with respect to the sprung band, each articulated segment including at least one emitter and being urged toward the scalp when the band is worn by the user.

29. (canceled)

30. The apparatus of claim 1 wherein the controller is operably configured to monitor the signal level produced at each detector and to control a level of infrared radiation produced by the selectively actuated emitter to maintain the intensity within a detection range of the detector;
optionally wherein the controller is further operably configured to generate display data for display as a graphic user interface (GUI) on a screen in communication with the controller, the GUI including a spatial representation of at least one of the emitters and detectors along with display information indicating whether the signal intensity is within the detection range of the associated detector;
or wherein the controller is operably configured to discontinue the monitoring when the signals received from the detectors no longer meet a coupling criterion indicative of a plurality of the emitters or detectors being coupled to the scalp of the user.

31-32. (canceled)

33. The apparatus of claim 28 further comprising at least one coupling sensor operably configured to generate a coupling signal indicating a state of coupling between the plurality of light pipes and the user's scalp, and wherein the controller is operably configured to discontinue the monitoring in response to the coupling signal indicating that a coupling criterion is not being met optionally
wherein the at least one coupling sensor comprises at least one of:
a capacitive sensor that produces a signal indicative of a proximity of the apparatus to the scalp;
an acoustic sensor that produces a signal in response to an ambient sound level;
an inertial sensor that produces a signal indicative of movement of the apparatus; or
one or more of the detectors, wherein an ambient light component in the signal produced by the one or more detectors is indicative of the apparatus being removed from the scalp and the detector being subject to ambient light radiation.

34. (canceled)

35. The apparatus of claim 30 wherein the controller is operably configured to process the signal received by at least one of the detectors to extract a cardiac pulse signal representing a detected heartbeat of the user and to monitor the pulse signal to determine whether coupling between the emitters and detectors and the scalp of the user meets a coupling criterion; optionally
wherein the controller is operably configured: to process the signals by extracting a dominant frequency from the signals that falls within a frequency range based on the user's expected heartbeat frequency range; and/or
to discontinue the monitoring when the cardiac pulse signals received from the detectors no longer meet the coupling criterion.

36-37. (canceled)

38. The apparatus of claim 1 wherein the controller is operably configured to monitor time variations in changes in blood oxygenation within the brain tissue in a region underlying each detector and selectively actuated emitter and to generate data metrics representing a degree of brain activation in each region; or
wherein the controller is further operably configured to generate display data for display as a graphic user interface (GUI) on a screen in communication with the controller, the GUI including a representation of regions of the user's body that correspond to regions of the user's brain that are indicated by the changes in blood oxygenation within the brain tissue to be actuated; or
wherein the controller comprises a processor circuit, the processor circuit including a graphic processing unit operably configured to accelerate processing of the signals from each of the plurality of detectors to facilitate near real time presentation of results to the user.

39-40. (canceled)

41. A method of measuring brain activity in a subject, said method comprising positioning the apparatus of claim 1 on the head of the subject; determining changes in blood oxygenation within the brain tissue and generating a spatial representation of brain activity within in the subject's brain based on said blood oxygenation within the brain tissue; optionally wherein said subject is the user of said apparatus.
